(19) United States
(12) Patent Application Publication
(10) Pub. No.: US 2026/0218172 A1
(43) Pub. Date: Jul. 30, 2026

(54) RNA GUIDED COMPOSITIONS FOR PREVENTING AND TREATING HEPATITIS B VIRUS INFECTIONS

(71) Applicant: Temple University - of the Commonwealth System of Higher Education, Philadelphia, PA (US)

(72) Inventors: Kamel Khalili, Bala Cynwyd, PA (US); Hassen Wollebo, Philadelphia, PA (US)

(21) Appl. No.: 19/404,723

(22) Filed: Dec. 1, 2025

Related U.S. Application Data
(63) Continuation of application No. 19/068,290, filed on Mar. 3, 2025, now abandoned.
Application No. 19/068,290 is a continuation of application No. 17/931,018, filed on Sep. 9, 2022, now abandoned.
Application No. 17/931,018 is a continuation of application No. 16/308,348, filed on May 26, 2017, now abandoned.
(60) Provisional application No. 62/346,859, filed on Jun. 7, 2016.
(60) Provisional application No. 62/474,912, filed on Mar. 22, 2017.

(51) Int. Cl. C12N 15/11; A61K 31/7088; A61K 38/46; A61P 31/20; C12N 9/22

(52) U.S. Cl. CPC C12N 15/11; A61K 31/7088; A61K 38/46; A61K 38/465; A61P 31/20; C12N 9/222; C12N 2310/20; C12N 2800/80

(57) ABSTRACT

Compositions that specifically cleave target sequences in Hepadnaviridae, for example Hepatitis B virus (HBV) include nucleic acids encoding a Clustered Regularly Interspaced Short Palindromic Repeat (CRISPR) associated endonuclease and a guide RNA sequence complementary to a target sequence in HBV. These compositions are administered to a subject for eradicating an infection, latent or otherwise, or at risk for contracting HBV infection.

### CROSS-REFERENCES TO RELATED APPLICATIONS

This application is a continuation of U.S. patent application Ser. No. 19/068,290, filed Mar. 3, 2025, which is a continuation of U.S. patent application Ser. No. 17/931,018, filed Sep. 9, 2022, which is a continuation of U.S. patent application Ser. No. 16/308,348, filed Dec. 17, 2018, which is the U.S. National Phase of PCT International Application No. PCT/US2017/034773, filed on May 26, 2017, which is an International Application of and claims the benefit of priority to U.S. Provisional Patent Application Nos. 62/474,912, filed on Mar. 22, 2017, and 62/346,859, filed Jun. 7, 2016, the entire contents of which are incorporated herein by reference in their entirety.

### INCORPORATION BY REFERENCE OF SEQUENCE LISTING

The contents of the sequence listing file named “Excision_SL_56852_718.301.xml”, which was created on Mar. 26, 2023 and is 50,507 bytes in size, are incorporated herein by reference in its entirety.

### FIELD OF THE INVENTION

Embodiments of the invention relate to compositions that specifically cleave target sequences in Hepadnaviridac, for example, hepatitis B virus (HBV). Such compositions, which include nucleic acids encoding a Clustered Regularly Interspaced Short Palindromic Repeat (CRISPR) associated endonuclease and a guide RNA sequence complementary to a target sequence in HBV, can be administered to a subject having or at risk for contracting an HBV infection.

### BACKGROUND

Viral hepatitis is the single most important cause of liver disease. Many infectious agents, including hepatitis A, B, C, D, and E viruses, can cause viral hepatitis. The Hepatitis B virus (HBV), for example, is a small, enveloped DNA virus that infects 400 million people worldwide. HBV is unusual among DNA viruses because its replication involves reverse transcription of an RNA intermediate. Infection with HBV induces a broad spectrum of liver diseases, including acute hepatitis (that can lead to fulminate hepatic failure) as well as chronic hepatitis, cirrhosis, and heptocellular carcinoma (HCC). There is an effective preventative vaccine, however, an estimated 280 million people are chronically infected with hepatitis B and more than 780,000 people die every year due to complications of hepatitis B, including cirrhosis and liver cancer (Lozano R. et al., Lancet 2012; 380:2095-2128).

### SUMMARY

Embodiments of the invention are directed, inter alia, to compositions for eradicating a hepadnavirus in vitro or in vivo. The compositions comprise, for example, a protein/nucleic acid or viral vector encoding a molecule which specifically targets Hepatitis B virus (HBV) and induces mutations and/or deletions in the viral DNA, rendering the DNA unable to undergo viral replication thus halting the viral life cycle and viral propagation.

In certain embodiments a composition comprises an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a hepadnavirus genome. In certain embodiments, a composition comprises two or three or four or more gRNAs. The gRNAs can target overlapping sequences, distinct target sequences or any combination of target sequences.

Other aspects are described infra.

### BRIEF DESCRIPTION OF THE DRAWINGS

FIG. 1A is a schematic representation showing a cocktail of gRNAs (SEQ ID NOS: 1-18) based on SaCas9 targeting P1, PS1 PS2, PS3 and X genes of HBV. Any one or more can be used to eradicate HBV in vivo or in vitro. FIG. 1B is a schematic representation showing the sequence and location in the HBV genome of the 12 candidate gRNAs designed by Benchling CRISPR design tool. The gRNAs are targeting five different genes: Pres1, Pres2, S, HBX and HBV Polymerase. Several gRNAs are designed to target different genotypes at the same position.

FIG. 2 shows a sequence of short hairpin RNA against Hepatitis B transactivator X. The shRNA targets and cleaves X gene mRNA through cellular RNA interference mechanisms.

FIG. 3 is a map of pX601-HBV3xgRNAs-shRNA construct targeting the Hepatitis B Virus genome. gRNA protospacer regions in red, shRNA for HBX in green, NLS-SaCas9-NLS-3xHA in brown-orange.

FIGS. 4A-4C are blots providing verification of the presence of gRNA/shRNA components in pX601-HBV3xgRNAs-shRNA plasmid. The presence of gRNAs expressing cassettes was checked in standard PCRs using U6 promoter forward and reverse primers specific to each of cloned gRNAs (FIG. 4A). Additionally, restriction digestion was performed using SacI/SpeI restriction enzymes to confirm existence of gRNAmotif2/motif3/shRNA insert upstream of SaCas9 gene (FIG. 4B). Finally, HBX shRNA presence was verified by XbaI/SpeI restriction digestion (FIG. 4C).

FIGS. 5A, 5B are blots providing confirmation of the correct SaCas9/gRNAs expression from pX601-HBV3xgRNAs-shRNA plasmid. TC620 cells were transfected with the final construct and 48 h later harvested for protein lysates and RNA. gRNAs expression was checked in reverse transcription followed by PCRs (FIG. 5A) using specific to each gRNA top oligonucleotides as a forward and gRNA scaffold as a reverse primer. NLS-SaCas9-NLS-3xHA protein expression was verified in Western blot using HA-tag antibody (FIG. 5B).

FIG. 6 shows the detection of the SaCas9/gRNAs induced excision of the HBV genome. The cleavage region was PCR amplified using two primers: forward, annealing 144 nucleotides upstream of the motif 1 and reverse, 191 downstream of motif 2 target site. Amplification using these primers yielded two products: full length 1454 bp long, representing the uncut/singly cut and end-joined HBV genomes and short 355 bp one corresponding to double cleaved/end joined viral sequences. The truncated double cleaved/end-joined band was purified from the gel, cloned and sent for Sanger sequencing. The obtained sequences were aligned using Clustal-Omega software using Hepatitis B genotype D sequence as a reference (FIG. 7). All clones showed perfect CRISPR/Cas9 mediated signature-cleavage three nucleotides from PAM at target sites for motifs 1 and 2. At the target motif 3 no any cleavage was detected sinee this gRNA was designed to targets exclusively HBV genotype A and in present in HepG2.2.15 HBV genotype D there are 5 mismatches at this target sites providing additional prove of SaCas9/gRNA specificity (FIG. 7).

FIG. 7 is a schematic representation showing the SaCas9/gRNA mediated excision of HBV sequences. The targeted region of HBV genome was PCR amplified and resolved in agarose gel. Truncated PCR products representing double cleaved/end-joined viral sequences (345 bp band) were purified, subcloned in TA vector and sequenced. Representative three truncated sequences are shown in relation to full length intact viral sequence as a reference. PCR primers are shown in green, target sequences in red followed my PAMs in yellow. The canonical, 3 nucleotides from PAM sequences, SaCas9/gRNAs mediated cleavage sites were detected with deletion of 1216 bp long viral DNA fragment between target sites motif 1 and 2. There was no cut at target site motif 3 sinee HBV genotype D present in HepG2.2.15 cells carries 5 mismatches in this region.

FIGS. 8A-8D show the analysis of the HBV genome cleavage efficiency in HepG2.2.15 cells. Cells were harvested at two timepoints: 3 and 7 days after transfection. Genomic DNA was prepared and analyzed in standard PCRs for detection of targeted region of HBV genome (FIG. 8A for 3 days and FIG. 8B for 7 days timepoint). To allow semi-quantification of excision efficiency, PCRs for human beta-actin were performed as a reference genomic DNA loading control for 7 days timepoint (FIG. 8B). The intensities of PCR bands from agarose gels were analyzed using ImageJ software (FIG. 8C) and plotted after normalizing to beta-actin levels (FIG. 8D).

FIG. 9 is a graph showing the quantification of intracellular HBV DNA levels in treated cells. Genomic DNA from transfected HepG2.2.15 cells was subjected to SYBRGREEN real time PCR reactions using primer sets specific to HBV pol and as a reference human beta-globin genes.

FIG. 10 is a graph showing the quantification of intracellular viral RNA levels. Total RNA was extracted from cells transfected with empty pX601 (SaCas9, no gRNA) and pX601-HBV3xgRNAs-shRNA (SaCas9 and gRNAs) at three days post-transfection and after one-week selection with puromycin. After reverse transcription using oligo-dT primers, SybrGreen real time PCRs were performed on diluted cDNA samples using primer sets specific to HBV pol and human beta-actin as a reference.

FIG. 11 is a graph showing the quantification of viral DNA levels in cell culture supernatants. Supernatants from transfected cells were precleared by centrifugation and heat deactivated to destroy infective viral particles. Next SybrGreen real time PCRs were performed on 10 times diluted in water samples using HBV X gene specific primers and standard prepared from serial dilutions of PCR amplification product corresponding to X gene of HBV.

### DETAILED DESCRIPTION

Embodiments of the invention are directed to compositions for eradicating a hepadnavirus, in vitro or in vivo. In particular, the compositions comprise isolated nucleic acid sequences encoding a Clustered Regularly Interspaced Short Palindromic Repeat (CRISPR)-associated endonuclease and at least one guide RNA (gRNA), the gRNA being complementary to a target nucleic acid sequence in a hepadnavirus genome, e.g. hepatitis B virus (HBV).

Hepatitis B is one of a few known pararetroviruses: non-retroviruses that still use reverse transcription in their replication process. The virus gains entry into the cell by binding to NTCP on the surface and being endocytosed. Because the virus multiplies via RNA made by a host enzyme, the viral genomic DNA has to be transferred to the cell nucleus by host proteins called chaperones. The partially double stranded viral DNA is then made fully double stranded by viral polymerase and transformed into covalently closed circular DNA (cccDNA). This cccDNA serves as a template for transcription of four viral mRNAs by host RNA polymerase. The largest mRNA, (which is longer than the viral genome), is used to make the new copies of the genome and to make the capsid core protein and the viral DNA polymerase. These four viral transcripts undergo additional processing and go on to form progeny virions that are released from the cell or returned to the nucleus and re-cycled to produce even more copies. The long mRNA is then transported back to the cytoplasm where the virion P protein (the DNA polymerase) synthesizes DNA via its reverse transcriptase activity.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

The articles “a” and “an” are used herein to refer to one or to more than one (i.e., to at least one) of the grammatical object of the article. By way of example, “an element” means one element or more than one element. Thus, recitation of “a cell”, for example, in includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “eradication” of the hepadnavirus, e.g. hepatitis B virus (HBV), as used herein, means that that virus is unable to replicate, the genome is deleted, fragmented, degraded, genetically inactivated, or any other physical, biological, chemical or structural manifestation, that prevents the virus from being transmissible or infecting any other cell or subject resulting in the clearance of the virus in vivo. In some cases, fragments of the viral genome may be detectable, however, the virus is incapable of replication, or infection etc.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having cither a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

The term “target nucleic acid” sequence refers to a nucleic acid (often derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

In the context of the present invention, the following abbreviations for the commonly occurring nucleic acid bases are used, “A” refers to adenosine, “C” refers to cytosine, “G” refers to guanosine, “T” refers to thymidine, and “U” refers to uridine.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The term “polynucleotide” is a chain of nucleotides, also known as a “nucleic acid”. As used herein polynucleotides include, but are not limited to, all nucleic acid sequences which are obtained by any means available in the art, and include both naturally occurring and synthetic nucleic acids.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “percent sequence identity” or having “a sequence identity” refers to the degree of identity between any given query sequence and a subject sequence.

The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

Where any amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Compositions for Eradication of Hepadnavirus in Cells or Subjects Hepatitis B virus (HBV) is a member of the Hepadnaviridae family (NCBI taxonomy). The virus particle (virion) consists of an outer lipid envelope and an icosahedral nucleocapsid core composed of protein. These virions are 30-42 nm in diameter. The nucleocapsid encloses the viral DNA and a DNA polymerase that has reverse transcriptase activity. The outer envelope contains embedded proteins that are involved in viral binding of, and entry into, susceptible cells. The virus is one of the smallest enveloped animal viruses, and the 42 nm virions are capable of infecting hepatocytes.

The virus is divided into four major serotypes (adr, adw, ayr, ayw) based on antigenic epitopes presented on its envelope proteins, and into eight genotypes (A-H) according to overall nucleotide sequence variation of the genome. The genotypes have a distinct geographical distribution and are used in tracing the evolution and transmission of the virus. Differences between genotypes affect the disease severity, course and likelihood of complications, and response to treatment and possibly vaccination. Genotypes differ by at least 8% of their sequence and were first reported in 1988 when six were initially described (A-F). Two further types have sinee been described (G and H). Most genotypes are now divided into subgenotypes with distinct properties.

HBV is an enveloped DNA virus that contains a small, partially double-stranded (DS), relaxed-circular DNA (rcDNA) genome that replicates by reverse transcription of an RNA intermediate, the pregenomic RNA (pgRNA). Its length is comprised between 3182 and 3248 bp depending on genotypes. The genome encodes four overlapping open reading frames (ORFs) that are translated into viral core protein, surface proteins, polymerase/reverse transcriptase (RT), and HBx.

One end of the full length strand is linked to the viral DNA polymerase. The negative-sense (non-coding) is complementary to the viral mRNA. The viral DNA is found in the nucleus soon after infection of the cell. The partially double-stranded DNA is rendered fully double-stranded by completion of the (+) sense strand and removal of a protein molecule from the (−) sense strand and a short sequence of RNA from the (+) sense strand. Non-coding bases are removed from the ends of the (−) sense strand and the ends are rejoined. There are four known genes encoded by the genome, called C, X, P, and S. The core protein is coded for by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S is the gene that codes for the surface antigen (HBsAg). The HBsAg gene is one long open reading frame but contains three in frame “start” (ATG) codons that divide the gene into three sections, pre-S1, pre-S2, and S. Because of the multiple start codons, polypeptides of three different sizes called large (the order from surface to the inside: pre-S1, pre-S2, and S), middle (pre-S2, S), and small(S) are produced. The function of the protein coded for by gene X is not fully understood but it is associated with the development of liver cancer. It stimulates genes that promote cell growth and inactivates growth regulating molecules. (Beck J., Nassal M. World J. Gastroenterol. 2007, 13 (1): 48-64; Seeger C., Mason W S. Microbiol. Mol. Rev. 200064 (1): 51-68; Urban S. et al., J. Hepatol. 2010, 52 (2): 282-284).

The HBV life cycle begins when the virus attaches to the host cell and is internalized. It has been demonstrated that sodium-taurocholate co-transporting polypeptide (NTCP) is a functional receptor in HBV infection (Yan H. et al., Elife, 2012, 00049). The virion rcDNA is delivered to the nucleus, where it is repaired to form a covalently closed-circular DNA (cccDNA). The episomal cccDNA serves as the template for the transcription of the pgRNA and the other viral mRNAs by the host RNA polymerase II. The transcripts are then exported to the cytoplasm, where translation of the viral proteins occurs. RT binds to pgRNA and triggers assembly of the core proteins into immature, RNA-containing nucleocapsids. The immature nucleocapsids then undergo a process of maturation whereby pgRNA is reversed transcribed by RT to make the mature rcDNA. A unique feature of hepadnavirus reverse transcription is the RT primed initiation of minus-strand DNA synthesis, which leads to the covalent linkage of RT to the 5′ end of the minus-strand DNA. (Nassal M. Virus Res. 2008, 216 (2): 282-284)

The mature, rcDNA-containing nucleocapsids are then enveloped by the viral surface proteins and secreted as virions (secretion pathway) or alternatively, are recycled back to the nucleus to further amplify the pool of cccDNA (recycling pathway). Persistence of cccDNA in hepatocytes plays a key role in viral persistence, reactivation of viral replication after cessation of antiviral therapy and resistance to therapy (Bruss V. Virus Res. 2004, 106 (2): 199-209; Nguyen D. H. et al., J. Physiol. 2008, 216 (2): 282-294).

Gene Editing Agents: Compositions of the invention include at least one gene editing agent, comprising CRISPR-associated nucleases such as Cas9 and Cpf1 gRNAs, Argonaute family of endonucleases, clustered regularly interspaced short palindromic repeat (CRISPR) nucleases, zinc-finger nucleases (ZFNs), transcription activator-like effector nucleases (TALENs), meganucleases, other endo- or exo-nucleases, or combinations thereof. See Schiffer, 2012, J Virol 88 (17): 8920-8936, incorporated by reference.

The composition can also include C2c2—the first naturally-occurring CRISPR system that targets only RNA. The Class 2 type VI-A CRISPR-Cas effector “C2c2” demonstrates an RNA-guided RNase function. C2c2 from the bacterium Leptotrichia shahii provides interference against RNA phage. In vitro biochemical analysis show that C2c2 is guided by a single crRNA and can be programmed to cleave ssRNA targets carrying complementary protospacers. In bacteria, C2c2 can be programmed to knock down specific mRNAs. Cleavage is mediated by catalytic residues in the two conserved HEPN domains, mutations in which generate catalytically inactive RNA-binding proteins. These results demonstrate the capability of C2c2 as a new RNA-targeting tools.

C2c2 can be programmed to cleave particular RNA sequences in bacterial cells. The RNA-focused action of C2c2 complements the CRISPR-Cas9 system, which targets DNA, the genomic blueprint for cellular identity and function. The ability to target only RNA, which helps carry out the genomic instructions, offers the ability to specifically manipulate RNA in a high-throughput manner- and manipulate gene function more broadly.

CRISPR/Cpf1 is a DNA-editing technology analogous to the CRISPR/Cas9 system, characterized in 2015 by Feng Zhang's group from the Broad Institute and MIT. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations. CRISPR/Cpf1 could have multiple applications, including treatment of genetic illnesses and degenerative conditions. As referenced above, Argonaute is another potential gene editing system.

Argonautes are a family of endonucleases that use 5′ phosphorylated short single-stranded nucleic acids as guides to cleave targets (Swarts, D. C. et al. The evolutionary journey of Argonaute proteins. Nat. Struct. Mol. Biol. 21, 743-753 (2014)). Similar to Cas9, Argonautes have key roles in gene expression repression and defense against foreign nucleic acids (Swarts, D. C. et al. Nat. Struct. Mol. Biol. 21, 743-753 (2014); Makarova, K. S., et al. Biol. Direct 4, 29 (2009). Molloy, S. Nat. Rev. Microbiol. 11, 743 (2013); Vogel, J. Science 344, 972-973 (2014). Swarts, D. C. et al. Nature 507, 258-261 (2014); Olovnikov, I., et al. Mol. Cell 51, 594-605 (2013)). However, Argonautes differ from Cas9 in many ways Swarts, D. C. et al. The evolutionary journey of Argonaute proteins. Nat. Struct. Mol. Biol. 21, 743-753 (2014)). Cas9 only exist in prokaryotes, whereas Argonautes are preserved through evolution and exist in virtually all organisms; although most Argonautes associate with single-stranded (ss) RNAs and have a central role in RNA silencing, some Argonautes bind ssDNAs and cleave target DNAs (Swarts, D. C. et al. Nature 507, 258-261 (2014); Swarts, D. C. et al. Nucleic Acids Res. 43, 5120-5129 (2015)). guide RNAs must have a 3′ RNA-RNA hybridization structure for correct Cas9 binding, whereas no specific consensus secondary structure of guides is required for Argonaute binding; whereas Cas9 can only cleave a target upstream of a PAM, there is no specific sequence on targets required for Argonaute. Once Argonaute and guides bind, they affect the physicochemical characteristics of each other and work as a whole with kinetic properties more typical of nucleic-acid-binding proteins (Salomon, W. E., et al. Cell 162, 84-95 (2015)).

Accordingly, in certain embodiments, Argonaute endonucleases comprise those which associate with single stranded RNA (ssRNA) or single stranded DNA (ssDNA). In certain embodiments, the Argonaute is derived from Natronobacterium gregoryi. In other embodiments, the Natronobacterium gregoryi Argonaute (NgAgo) is a wild type NgAgo, a modified NgAgo, or a fragment of a wild type or modified NgAgo. The NgAgo can be modified to increase nucleic acid binding affinity and/or specificity, alter an enzymatic activity, and/or change another property of the protein. For example, nuclease (e.g., DNasc) domains of the NgAgo can be modified, deleted, or inactivated.

The wild type NgAgo sequence can be modified. The NgAgo nucleotide sequence can be modified to encode biologically active variants of NgAgo, and these variants can have or can include, for example, an amino acid sequence that differs from a wild type NgAgo by virtue of containing one or more mutations (e.g., an addition, deletion, or substitution mutation or a combination of such mutations). One or more of the substitution mutations can be a substitution (e.g., a conservative amino acid substitution). For example, a biologically active variant of an NgAgo polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type NgAgo polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the NgAgo amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine (2R,3S)-2-amino-3-methylpentanoic acid and Lcyclopentyl glycine(S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

Another gene editing agent is human WRN, a RecQ helicase encoded by the Werner syndrome gene. It is implicated in genome maintenance, including replication, recombination, excision repair and DNA damage response. These genetic processes and expression of WRN are concomitantly upregulated in many types of cancers. Therefore, it has been proposed that targeted destruction of this helicase could be useful for elimination of cancer cells. Reports have applied the external guide sequence (EGS) approach in directing an RNase P RNA to efficiently cleave the WRN mRNA in cultured human cell lines, thus abolishing translation and activity of this distinctive 3′-5′ DNA helicase-nuclease. RNase P RNA are another potential endonuclease for use with the present invention.

CRISPR-Associated Endonucleases: In embodiments, the compositions disclosed herein, include nucleic acids encoding a CRISPR-associated endonuclease, such as Cas9. In some embodiments, one or more guide RNAs that are complementary to a target sequence of a hepadnavirus may also be encoded.

In general, CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNase domains, protein-protein interaction domains, dimerization domains, as well as other domains.

CRISPR (Clustered Regularly Interspaced Short Palindromic Repeats) is found in bacteria and is believed to protect the bacteria from phage infection. It has recently been used as a means to alter gene expression in eukaryotic DNA, but has not been proposed as an anti-viral therapy or more broadly as a way to disrupt genomic material. Rather, it has been used to introduce insertions or deletions as a way of increasing or decreasing transcription in the DNA of a targeted cell or population of cells. See for example, Horvath et al., Science (2010) 327:167-170; Terns et al., Current Opinion in Microbiology (2011) 14:321-327; Bhaya et al., Annu Rev Genet (2011) 45:273-297; Wiedenheft et al., Nature (2012) 482:331-338); Jinek M et al., Science (2012) 337:816-821; Cong L et al., Science (2013) 339:819-823; Jinek M et al., (2013) eLife 2: e00471; Mali P et al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918).

CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligonucleotide to hybridize to target and recruit the Cas/gRNA complex. See Chang et al., 2013, Cell Res. 23:465-472; Hwang et al., 2013, Nat. Biotechnol. 31:227-229; Xiao et al., 2013, Nucl. Acids Res. 1-11.

In general, the CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNase domains, protein-protein interaction domains, dimerization domains, as well as other domains. The mechanism through which CRISPR/Cas9-induced mutations inactivate the provirus can vary. For example, the mutation can affect proviral replication, and viral gene expression. The mutation can comprise one or more deletions. The size of the deletion can vary from a single nucleotide base pair to about 10,000 base pairs. In some embodiments, the deletion can include all or substantially all of the proviral sequence. In some embodiments the deletion can eradicate the provirus. The mutation can also comprise one or more insertions, that is, the addition of one or more nucleotide base pairs to the proviral sequence. The size of the inserted sequence also may vary, for example from about one base pair to about 300 nucleotide base pairs. The mutation can comprise one or more point mutations, that is, the replacement of a single nucleotide with another nucleotide. Useful point mutations are those that have functional consequences, for example, mutations that result in the conversion of an amino acid codon into a termination codon, or that result in the production of a nonfunctional protein.

In embodiments, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In some embodiments, the CRISPR/Cas-like protein can be derived from a wild type Cas9 protein or fragment thereof. In other embodiments, the CRISPR/Cas-like protein can be derived from modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

Three types (I-III) of CRISPR systems have been identified. CRISPR clusters contain spacers, the sequences complementary to antecedent mobile elements. CRISPR clusters are transcribed and processed into mature CRISPR RNA (crRNA). In embodiments, the CRISPR/Cas system can be a type I, a type II, or a type III system. Non-limiting examples of suitable CRISPR/Cas proteins include Cas3, Cas4, Cas5, Casse (or CasD), Cas6, Casde, Cas6f, Cas7, Cas8al, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx 14, Csx10, Csx16, CsaX, Csx3, Csz1, Csx15, Csf1, Csf2, Csf3, Csf4, and Cu1966.

In one embodiment, the RNA-guided endonuclease is derived from a type II CRISPR/Cas system. The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA: tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately.

The CRISPR-associated endonuclease Cas9 nuclease can have a nucleotide sequence identical to the wild type Streptococcus pyogenes sequence. The CRISPR-associated endonuclease may be a sequence from other species, for example other Streptococcus species, such as thermophiles. The Cas9 nuclease sequence can be derived from other species including, but not limited to: Nocardiopsis dassonvillei, Streptomyces pristinaespiralis, Streptomyces viridochromogenes, Streptomyces roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficle, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Pseudomonas aeruginosa, Escherichia coli, or other sequenced bacteria genomes and archaea, or other prokaryotic microorganisms may also be a source of the Cas9 sequence utilized in the embodiments disclosed herein.

The wild type Streptococcus pyogenes Cas9 sequence can be modified. The nucleic acid sequence can be codon optimized for efficient expression in mammalian cells, i.e., “humanized”. The Cas9 sequence can be for example, the Cas9 nuclease sequence encoded by any of the expression vectors listed in Genbank accession numbers KM099231.1 GI: 669193757; KM099232.1 GI: 669193761; or KM099233.1 GI: 669193765. Alternatively, the Cas9 nuclease sequence can be for example, the sequence contained within a commercially available vector such as PX330 or PX260 from Addgene (Cambridge, MA). In some embodiments, the Cas9 endonuclease can have an amino acid sequence that is a variant or a fragment of any of the Cas9 endonuclease sequences of Genbank accession numbers KM099231.1 GI: 669193757; KM099232.1 GI: 669193761; or KM099233.1 GI: 669193765 or Cas9 amino acid sequence of PX330 or PX260 (Addgene, Cambridge, MA). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucinc (2R,3S)-2-amino-3-methylpentanoic acid and L-cyclopentyl glycine(S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

The Cas9 nuclease sequence can be a mutated sequence. For example, the Cas9 nuclease can be mutated in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks.

The Cas9 can be an orthologous. Six smaller Cas9 orthologues have been used and reports have shown that Cas9 from Staphylococcus aureus (SaCas9) can edit the genome with efficiencies similar to those of SpCas9, while being more than 1 kilobase shorter.

In addition to the wild type and variant Cas9 endonucleases described, embodiments of the invention also encompass CRISPR systems including newly developed “enhanced-specificity” S. pyogenes Cas9 variants (eSpCas9), which dramatically reduce off target cleavage. These variants are engineered with alanine substitutions to neutralize positively charged sites in a groove that interacts with the non-target strand of DNA. This aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOI: 10.1126/science.aad5227).

In certain embodiments, three variants found to have the best cleavage efficiency and fewest off-target effects: SpCas9 (K855A), SpCas9 (K810A/K1003A/R1060A) (a.k.a. eSpCas9 1.0), and SpCas9 (K848A/K1003A/R1060A) (a.k.a. eSPCas9 1.1) are employed in the compositions. The invention is by no means limited to these variants, and also encompasses all Cas9 variants (Slaymaker, I. M. et al. (2015)).

The present invention also includes another type of enhanced specificity Cas9 variant, “high fidelity” spCas9 variants (HF-Cas9) (Kleinstiver, B. P. et al., 2016, Nature. DOI: 10.1038/nature16526).

As used herein, the term “Cas” is meant to include all Cas molecules comprising variants, mutants, orthologues, high-fidelity variants and the like.

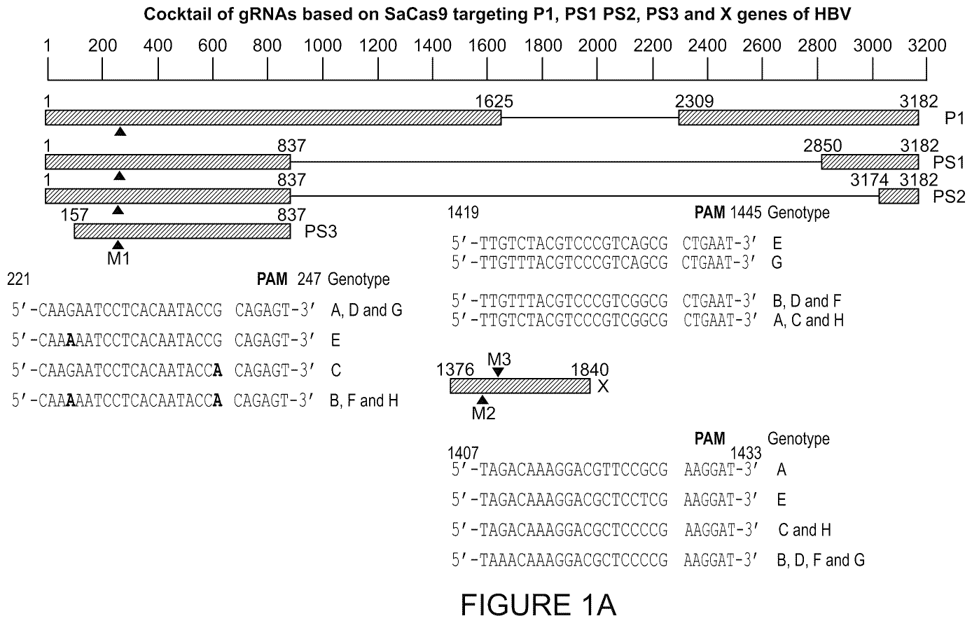

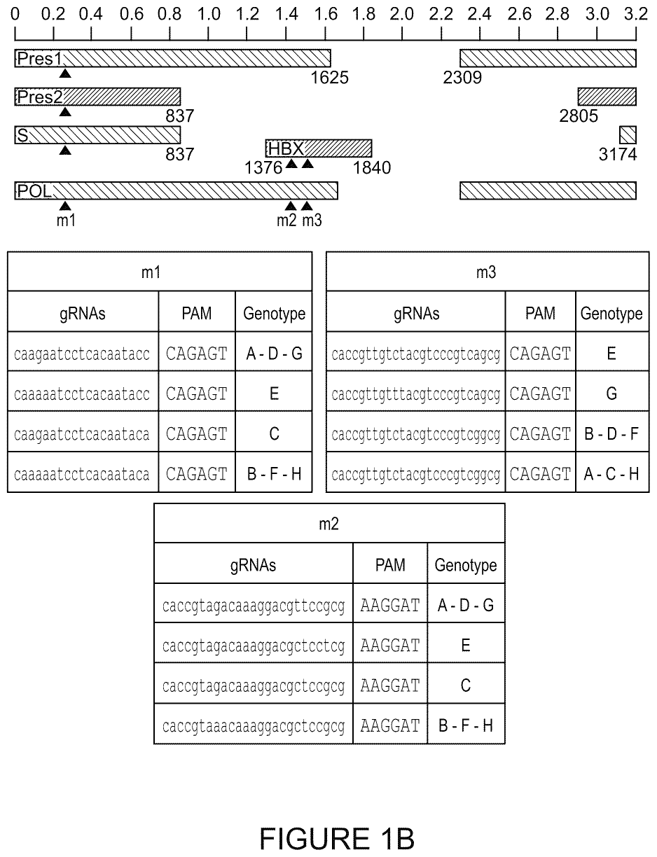

Guide Nucleic Acid Sequences: Guide RNA sequences according to the present invention can be sense or anti-sense sequences. The guide RNA sequence generally includes a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. For example, Cas9 from S. thermophilus requires 5′-NNAGAA for CRISPR 1 and 5′-NGGNG for CRISPR3 and Neiseria meningitidis requires 5′-NNNNGATT. PAM sequences are also shown in FIGS. 1A, 1B. The specific sequence of the guide RNA may vary, but, regardless of the sequence, useful guide RNA sequences will be those that minimize off-target effects while achieving high efficiency and complete ablation of the hepadnavirus, for example, HBV. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs.

The compositions and methods of the present invention may include a sequence encoding a guide RNA that is complementary to a target sequence in a hepadnavirus. In one embodiment, the hepadnavirus is HBV.

In certain embodiments, a composition for eradicating a hepadnavirus in vitro or in vivo, comprises an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a hepadnavirus genome. In certain embodiments, a composition comprises two or three or four or more gRNAs. The gRNAs can target overlapping sequences, distinct sequences or any combination of target sequences. For example, the two or more gRNAs comprise two or more nucleic acid sequences comprising SEQ ID NOS: 1-18.

In certain embodiments, composition for eradicating a hepadnavirus in vitro or in vivo, comprises: an isolated nucleic acid sequence encoding a Clustered Regularly Interspaced Short Palindromic Repeat (CRISPR)-associated endonuclease and two or more guide RNAs (gRNAs), the gRNAs being complementary to a target nucleic acid sequence in a hepadnavirus genome. In certain embodiments, a composition comprises two or three or four or more gRNAs. The gRNAs can target overlapping sequences, distinct sequences or any combination of target sequences. For example, the gRNAs comprise two or more nucleic acid sequences comprising SEQ ID NOS: 1-18.

In another embodiment, a target nucleic acid sequence comprises one or more nucleic acid sequences in coding and non-coding nucleic acid sequences of the hepadnavirus genome. The target nucleic acid sequence can be located within a sequence encoding structural proteins, non-structural proteins or combinations thereof. For example, the HBV sequences encoding structural and non-structural proteins comprise C, X, P, and S nucleic acid sequences. In certain embodiments, the gRNAs are designed to target P1, PS1, PS2, PS3 and X genes of HBV.

In certain embodiments, a gRNA sequence has at least a 75% sequence identity to target nucleic acid sequences comprising C, X, P, and S nucleic acid sequences, or combinations thereof. In other embodiments, a gRNA sequence has at least a 75% sequence identity to target nucleic acid sequences comprising P1, PS1, PS2, PS3 and X nucleic acid sequences, or combinations thereof.

Non-limiting examples of gRNA nucleic acid sequences are shown in FIG. 1A, 1B and are as follows:

(SEQ ID NO: 1)
5′-CAAGAATCCTCACAATACCG-3′;
(SEQ ID NO: 2)
5′-CAAAAATCCTCACAATACCG-3′;
(SEQ ID NO: 3)
5′-CAAGAATCCTCACAATACCA-3′;
(SEQ ID NO: 4)
5′-CAAAAATCCTCACAATACCA-3′;
(SEQ ID NO: 5)
5′-TTGTCTACGTCCCGTCAGCG-3′;
(SEQ ID NO: 6)
5′-TTGTTTACGTCCCGTCAGCG-3′;
(SEQ ID NO: 7)
5′-TTGTTTACGTCCCGTCGGCG-3′;
(SEQ ID NO: 8)
5′-TTGTCTACGTCCCGTCGGCG-3′;
(SEQ ID NO: 9)
5′-TAGACAAAGGACGTTCCGCG-3′;
(SEQ ID NO: 10)
5′-TAGACAAAGGACGCTCCTCG-3′;
(SEQ ID NO: 11)
5′-TAGACAAAGGACGCTCCCCG-3′;
(SEQ ID NO: 12)
5′-TAAACAAAGGACGCTCCCCG-3′.

In other embodiments, the gRNA sequences have at least a 75% sequence identity to sequences comprising: SEQ ID NOS: 1-18, or combinations thereof. In other embodiments, the gRNA sequences comprise: SEQ ID NOS: 1-18, or combinations thereof.

In other embodiments, the gRNA sequences have at least a 50% sequence identity to sequences comprising: SEQ ID NOS: 1-30, or combinations thereof. In other embodiments, the gRNA sequences comprise: SEQ ID NOS: 1-30, or combinations thereof.

In other embodiments, an isolated nucleic acid sequence comprises at least a 50% sequence identity to one or more sequences comprising SEQ ID NOS: 1 to 30. In other embodiments, the isolated nucleic acid sequences comprise any one or more of SEQ ID NOS: 1-30.

In certain embodiments, an isolated nucleic acid sequence comprises a nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a hepadnavirus genome.

When the compositions are administered as a nucleic acid or are contained within an expression vector, the CRISPR endonuclease can be encoded by the same nucleic acid or vector as the guide RNA sequences. Alternatively, or in addition, the CRISPR endonuclease can be encoded in a physically separate nucleic acid from the gRNA sequences or in a separate vector.

Modified or Mutated Nucleic Acid Sequences: In some embodiments, any of the nucleic acid sequences may be modified or derived from a native nucleic acid sequence, for example, by introduction of mutations, deletions, substitutions, modification of nucleobases, backbones and the like. The nucleic acid sequences include the vectors, gene-editing agents, gRNAs, tracrRNA etc. Examples of some modified nucleic acid sequences envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH₂—NH—O—CH₂, CH₂—N(CH₃)—O—CH₂ [known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH). The amide backbones disclosed by De Mesmaeker et al. Acc. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

The nucleic acid sequences may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino) adenine, 2-(imidazolylalkyl) adenine, 2-(aminoalklyamino) adenine or other heterosubstituted alkyladenines, 2-thiouracil, 2-thiothymine, 5-bromouracil, 5-hydroxymethyluracil, 8-azaguanine, 7-deazaguanine, N₆(6-aminohexyl) adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278).

Another modification of the nucleic acid sequences of the invention involves chemically linking to the nucleic acid sequences one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651).

It is not necessary for all positions in a given nucleic acid sequence to be uniformly modified, and in fact more than one of the aforementioned modifications may be incorporated in a single nucleic acid sequence or even at within a single nucleoside within a nucleic acid sequence.

In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Bonne, cds. (1998, ASM Press, Washington DC). Modified RNA components include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl) uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl) uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N.sup.6Nmethyladenosine; N⁶, N⁶-dimethyladenosine; N⁶,2′-O-trimethyladenosine; 2 methylthio-N⁶Nisopentenyladenosine; N⁶-(cis-hydroxyisopentenyl)-adenosine; 2-methylthio-N⁶-(cis-hydroxyisopentenyl)-adenosine; N⁶-glycinylcarbamoyl) adenosine; N⁶threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1;2′-O-dimethyl inosine; 2′-O-methyl guanosine; 1-methyl guanosine; N²-methyl guanosine; N², N²-dimethyl guanosine; N², 2′-O-dimethyl guanosine; N², N², 2′-O-trimethyl guanosine; 2′-O-ribosyl guanosine (phosphate); 7-methyl guanosine; N²;7-dimethyl guanosine; N²; N²;7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

The isolated nucleic acid molecules of the present invention can be produced by standard techniques. For example, polymerase chain reaction (PCR) techniques can be used to obtain an isolated nucleic acid containing a nucleotide sequence described herein. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

### Delivery Vehicles

Delivery vehicles as used herein, include any types of molecules for delivery of the compositions embodied herein, both for in vitro or in vivo delivery. Examples, include, without limitation: expression vectors, nanoparticles, colloidal compositions, lipids, liposomes, nanosomes, carbohydrates, organic or inorganic compositions and the like.

In some embodiments, a delivery vehicle is an expression vector, wherein the expression vector comprises an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a hepadnavirus genome.

Nucleic acids as described herein may be contained in vectors. Vectors can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FLAG™ tag (Kodak, New Haven, CT) sequences typically are expressed as a fusion with the encoded polypeptide. Such tags can be inserted anywhere within the polypeptide, including at cither the carboxyl or amino terminus.

Additional expression vectors also can include, for example, segments of chromosomal, non-chromosomal and synthetic DNA sequences. Suitable vectors include derivatives of SV40 and known bacterial plasmids, e.g., E. coli plasmids col EL, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2 μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

Several delivery methods may be utilized in conjunction with the isolated nucleic acid sequences for in vitro (cell cultures) and in vivo (animals and patients) systems. In one embodiment, a lentiviral gene delivery system may be utilized. Such a system offers stable, long term presence of the gene in dividing and non-dividing cells with broad tropism and the capacity for large DNA inserts. (Dull et al, J Virol, 72:8463-8471 1998).

In an embodiment, adeno-associated virus (AAV) may be utilized as a delivery method. AAV is a non-pathogenic, single-stranded DNA virus that has been actively employed in recent years for delivering therapeutic gene in in vitro and in vivo systems (Choi et al, Curr Gene Ther, 5:299-310, 2005). As an example, a non-viral delivery method may utilize nanoparticle technology. This platform has demonstrated utility as a pharmaceutical in vivo. Nanotechnology has improved transcytosis of drugs across tight epithelial and endothelial barriers. It offers targeted delivery of its payload to cells and tissues in a specific manner (Allen and Cullis, Science, 303:1818-1822, 1998).

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

The term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses Ad, AAV, lentivirus, and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34:167-171 (2003). A large variety of such vectors are known in the art and are generally available. A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88:8850-8854, 1991).

Additional vectors include viral vectors, fusion proteins and chemical conjugates. Retroviral vectors include Moloney murine leukemia viruses and HIV-based viruses. One HIV based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector [Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.: 90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3:219 (1993); Yang, et al., J. Virol. 69:2004 (1995)] and Adeno-associated Virus Vectors [Kaplitt, M. G., et al., Nat. Genet. 8:148 (1994)].

The polynucleotides disclosed herein may be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11 (2): 21 (1989) and Maurer, R. A., Bethesda Res. Lab. Focus, 11 (2): 25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another delivery method is to use single stranded DNA producing vectors which can produce the expressed products intracellularly. See for example, Chen et al, BioTechniques, 34:167-171 (2003), which is incorporated herein, by reference, in its entirety.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are commonly latently infected reservoirs of HBV infection, for example, brain macrophages, microglia, astrocytes, and gut-associated lymphoid cells. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding an isolated nucleic acid sequence comprising a sequence encoding a CRISPR-associated endonuclease and a guide RNA complementary to a target sequence of HBV, as described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol modified (PEGylated) low molecular weight LPEI.

The nucleic acids and vectors may also be applied to a surface of a device (e.g., a catheter) or contained within a pump, patch, or other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments, the compositions can be formulated as a nanoparticle encapsulating the compositions embodied herein.

Regardless of whether compositions are administered as nucleic acids or polypeptides, they are formulated in such a way as to promote uptake by the mammalian cell. Useful vector systems and formulations are described above. In some embodiments the vector can deliver the compositions to a specific cell type. The invention is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems.

In other embodiments, the compositions comprise a cell which has been transformed or transfected with one or more Cas/gRNA vectors. In some embodiments, the methods of the invention can be applied ex vivo. That is, a subject's cells can be removed from the body and treated with the compositions in culture to excise, for example, HBV sequences and the treated cells returned to the subject's body. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are self-derived cell, make it possible to avoid rejection reactions, which are the biggest obstacle to regenerative medicine or transplantation therapy.

The isolated nucleic acids can be easily delivered to a subject by methods known in the art, for example, methods which deliver siRNA. In some aspects, the Cas may be a fragment wherein the active domains of the Cas molecule are included, thereby cutting down on the size of the molecule. Thus, the, Cas9/gRNA molecules can be used clinically, similar to the approaches taken by current gene therapy. In particular, a Cas9/multiplex gRNA stable expression stem cell or iPS cells for cell transplantation therapy as well as vaccination can be developed for use in subjects.

Transduced cells are prepared for reinfusion according to established methods. After a period of about 2-4 weeks in culture, the cells may number between 1×10⁶ and 1×10¹⁰. In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent. For administration, cells of the present invention can be administered at a rate determined by the LD₅₀ of the cell type, and the side effects of the cell type at various concentrations, as applied to the mass and overall health of the patient. Administration can be accomplished via single or divided doses. Adult stem cells may also be mobilized using exogenously administered factors that stimulate their production and egress from tissues or spaces that may include, but are not restricted to, bone marrow or adipose tissues.

### Methods of Treatment

In certain embodiments, a method of eradicating a hepadnavirus genome in a cell or a subject, comprises contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a hepadnavirus genome.

In other embodiments, a method of inhibiting replication of a hepadnavirus in a cell or a subject, comprising contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a hepadnavirus genome.

The compositions of the present invention can be prepared in a variety of ways known to one of ordinary skill in the art. Regardless of their original source or the manner in which they are obtained, the compositions disclosed herein can be formulated in accordance with their use. For example, the nucleic acids and vectors described above can be formulated within compositions for application to cells in tissue culture or for administration to a patient or subject. Any of the pharmaceutical compositions of the invention can be formulated for use in the preparation of a medicament, and particular uses are indicated below in the context of treatment, e.g., the treatment of a subject having a hepatitis B viral infection or at risk for contracting a hepatitis B virus infection.

When employed as pharmaceuticals, any of the nucleic acids and vectors can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

The pharmaceutical compositions may contain, as the active ingredient, nucleic acids and vectors described herein in combination with one or more pharmaceutically acceptable carriers. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

An effective amount of any composition provided herein can be administered to an individual in need of treatment. An effective amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

Dosage, toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As described, a therapeutically effective amount of a composition (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

In certain embodiments, the anti-viral agent comprises therapeutically effective amounts of: antibodies, aptamers, adjuvants, anti-sense oligonucleotides, chemokines, cytokines, immune stimulating agents, immune modulating molecules, B-cell modulators, T-cell modulators, NK cell modulators, antigen presenting cell modulators, enzymes, siRNA's, interferon, ribavirin, ribozymes, protease inhibitors, anti-sense oligonucleotides, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, vaccines or combinations thereof.

The immune-modulating molecules comprise, but are not limited to cytokines, lymphokines, T cell co-stimulatory ligands, etc. An immune-modulating molecule positively and/or negatively influences the humoral and/or cellular immune system, particularly its cellular and/or non-cellular components, its functions, and/or its interactions with other physiological systems. The immune-modulating molecule may be selected from the group comprising cytokines, chemokines, macrophage migration inhibitory factor (MIF; as described, inter alia, in Bernhagen (1998), Mol Med 76 (3-4); 151-61 or Metz (1997), Adv Immunol 66, 197-223), T-cell receptors or soluble MHC molecules. Such immune-modulating effector molecules are well known in the art and are described, inter alia, in Paul, “Fundamental immunology”, Raven Press, New York (1989). In particular, known cytokines and chemokines are described in Meager, “The Molecular Biology of Cytokines” (1998), John Wiley & Sons, Ltd., Chichester, West Sussex, England; (Bacon (1998). Cytokine Growth Factor Rev 9 (2): 167-73; Oppenheim (1997). Clin Cancer Res 12, 2682-6; Taub, (1994) Ther. Immunol. 1 (4), 229-46 or Michiel, (1992). Semin Cancer Biol 3 (1), 3-15).

Immune cell activity that may be measured include, but is not limited to, (1) cell proliferation by measuring the DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

Also of interest are enzymes present in the lytic package that cytotoxic T lymphocytes or LAK cells deliver to their targets. Perforin, a pore-forming protein, and Fas ligand are major cytolytic molecules in these cells (Brandau et al., Clin. Cancer Res. 6:3729, 2000; Cruz et al., Br. J. Cancer 81:881, 1999). CTLs also express a family of at least 11 serine proteases termed granzymes, which have four primary substrate specificities (Kam et al., Biochim. Biophys. Acta 1477:307, 2000). Low concentrations of streptolysin O and pneumolysin facilitate granzyme B-dependent apoptosis (Browne et al., Mol. Cell Biol. 19:8604, 1999).

Other suitable effectors encode polypeptides having activity that is not itself toxic to a cell, but renders the cell sensitive to an otherwise nontoxic compound—either by metabolically altering the cell, or by changing a non-toxic prodrug into a lethal drug. Exemplary is thymidine kinase (tk), such as may be derived from a herpes simplex virus, and catalytically equivalent variants. The HSV tk converts the anti-herpetic agent ganciclovir (GCV) to a toxic product that interferes with DNA replication in proliferating cells.

In certain embodiments, the antiviral agent comprises natural or recombinant interferon-alpha (IFNα), interferon-beta (IFNβ), interferon-gamma (IFNγ), interferon tau (IFNτ), interferon omega (IFNω), or combinations thereof. In some embodiments, the interferon is IFNγ. Any of these interferons can be stabilized or otherwise modified to improve the tolerance and biological stability or other biological properties. One common modification is pegylation (modification with polyethylene glycol).

### Kits

The compositions described herein can be packaged in suitable containers labeled, for example, for use as a therapy to treat a subject having a hepadnavirus infection, for example, a hepatitis B virus infection or a subject at risk of contracting a hepatitis B virus infection. The containers can include a composition comprising a nucleic acid sequence, e.g. an expression vector encoding a CRISPR-associated endonuclease, for example, a Cas9 endonuclease, and a guide RNA complementary to a target sequence in a hepadnavirus, or a vector encoding that nucleic acid, and one or more of a suitable stabilizer, carrier molecule, flavoring, and/or the like, as appropriate for the intended use. Accordingly, packaged products (e.g., sterile containers containing one or more of the compositions described herein and packaged for storage, shipment, or sale at concentrated or ready-to-use concentrations) and kits, including at least one composition of the invention, e.g., a nucleic acid sequence encoding a CRISPR-associated endonuclease, for example, a Cas9 endonuclease, and a guide RNA complementary to a target sequence in HBV, or a vector encoding that nucleic acid and instructions for use, are also within the scope of the invention. A product can include a container (e.g., a vial, jar, bottle, bag, or the like) containing one or more compositions of the invention. In addition, an article of manufacture further may include, for example, packaging materials, instructions for use, syringes, delivery devices, buffers or other control reagents for treating or monitoring the condition for which prophylaxis or treatment is required.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions can be provided in a concentrated form with a diluent and instructions for dilution.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, applicants do not admit any particular reference is “prior art” to their invention.

### EXAMPLES

### Example 1: CRISPR/SaCas9-Based HBV Therapy

### Materials and Methods

Cloning of CRISPR/SaCas9 Constructs. To create the all-in-one SaCas9/gRNA/shRNA construct targeting HBV genome, the existing pX601-AAV-CMV::NLS-SaCas9-NLS-3xHA-bGHpA; U6::Bsa1-sgRNA plasmid was used (Addgene #61591) consisting of Staphylococcus aureus derived SaCas9/gRNA system adapted for use in mammalian cells. Protospacer regions corresponding to selected target sites were ordered as pairs of 5′-G (N¹⁹)-3′ complementary oligonucleotides containing BsaI overhangs at their respective 5′ ends (Table 1). After annealing and phosphorylation using T4 polynucleotide kinase (NEB) double stranded protospacers were ligated into BsaI digested, dephosphorylated with Calf Intestine Phosphatase (CIP, NEB) pX601 backbone plasmid. Bacterial clones were screened for the presence of gRNA protospacer inserts by PCRs using top, forward gRNA oligonucleotides in combination with reverse primer from scaffold gRNA segment of U6-gRNA cassette (Table 1). Successful clones were further verified by sequencing using the same reverse primer. To create HBV3xgRNA construct, motif 2 and 3 gRNA expressing cassettes were PCR amplified from their respective pX601 plasmids using primers containing XbaI (in forward) and SpeI (in reverse) restriction sites and ligated into XbaI digested pX601-HBVmotif1 plasmid in two cycles of XbaI restriction digestion/ligation. In final step, to add to the construct HBV X shRNA expressing cassette, XbaI/SpeI extended oligonucleotides containing minimal 24 bp U6 promoter allowing direct cloning of annealed double stranded hairpin coding sequence into XbaI digested pX601-HBV3xgRNAs plasmid resulting in pX601-HBV3xgRNAs/shRNA vector.

Cell Culture: HepG2.2.15 and TC120 cell line cells were cultured in Dulbecco's Modified Eagle's Medium (DMEM) (Life Technologies, NY) supplemented with 10% fetal bovine serum (FBS), 2 mM glutamine and 400 μg/ml of Gentamycin (Life Technologies, NY). To promote cell attachment all culture dishes and plates were precoated with poly-D-lysine prior plating cells. For puromycin selection cells were incubated in growth medium containing 3 μg/ml of puromycin (Sigma Aldrich). Medium was changed every day for one week to achieve maximum selection strength.

Antibodies. To detect NLS-SaCas9-NLS-3xHA, HA-tag antibody was used (1:1000, Abcam) for Western blot loading control anti-tubulin clone B512 from (1:5000, Sigma Aldrich).

Transfection. Cells were plated in 6 well plates at density 150000 cells per well. Next day cells were transfected using Lipofectamine 2000 reagent (Invitrogen) according manufacturer protocol. Briefly, 7.5 μl Lipofectamine 2000 was resuspended in 100 μl of Opti-MEM medium (Gibco) and incubated for 5 minutes. Meantime plasmid DNA mixtures were prepared: 2 μg of control empty pX601 or pX601-HBV3xgRNAs/shRNA together with 0.5 μg of pKLV-U6gRNA (BbsI)-PGKpuro2ABFP (Addgene #50946, to provide puromycin resistance for selection and BFP for transfection efficiency control) plasmids were added to 100 μl of Opti-MEM medium mixed and then combined with 100 μl Lipofectamine 2000/Opti-MEM and incubated for 15 minutes at room temperature (DNA: lipofectamine ratio: 1:2.5). Next DNA/Lipofectamine complexes (200 μl) were vortexed and added dropwise into 800 μl Opti-MEM per well in culture plates. After 4 hours incubation 1 ml/well of growth medium was added and left overnight. Next day, medium was replaced with fresh growth medium and cells were incubated for another 48 h before harvesting.

Viral DNA extraction and analysis. Cell pellets were collected and DNA was extracted using NUCLEOSPIN kit (Macherey-Nagel) according to the manufacturer's protocol, and the final product was eluted in 60 μl of water. For standard PCRs, 250 ng of genomic DNA was used. Reaction mixtures were prepared using FAIL SAFE Kit enzyme mix, PCR buffer J (Epicenter) and primers designed to amplify the targeted region of HBV genotype D (see Table 1). Quantification of HBV intracellular DNA was performed with 50 ng of genomic DNA per well using SYBRGREEN real time PCR (Roche) with primer sets specific to pol and X viral genes and human beta-globin as a reference (Table 1).

Analysis of RNA. Total RNA was extracted from cell pellets using RNAesy kit (Qiagen) according manufacturer protocol. Next 2.5 μg of RNA was used for reverse transcription reactions using M-MLV reverse transcription (Invitrogen) and different reverse primers depending on the purpose of experiment. For detection/verification of gRNAs expression in transfected cells, gRNA scaffold reverse primer was used (Table 1) followed by standard PCR using top gRNA specific oligonucleotide as a forward primer and the same gRNA scaffold reverse primer. In case of quantification of intracellular viral RNA levels, oligo-dT primer mix was utilized in reverse transcription and primer sets specific to viral polymerase and reference human beta-actin (Table 1) were used in SYBRGREEN real time PCR reactions (Roche).

Quantification of virus level in cell culture supernatants. SYBRGREEN real time PCR was used to quantify viral DNA levels in supernatants of infected cells. Culture medium was collected and spun down for 10 minutes at 3000 RPM to remove floating cells and cell debris. Next supernatants were incubated for 5 minutes at 95° C. to denature/destroy infective viral particles. A standard curve was prepared using serial dilutions of PCR amplified fragment of HBV genome spanning core and X genes (primers, Table 1). qPCR reactions were performed using 5 μl of deactivated, ten times diluted in water supernatants and HBV X specific primers.

CRISPR/Cas9 design and validation. Using the CRISPR online design tool available on (benchling.com), 12 single guide RNAs (sgRNAs) were generated, targeting the HBV genome (FIGS. 1A, 1B). Target sequences were chosen in order to maximize conservation across viral genotypes, and minimize homology to the human genome. Based on these criteria, only guides targeting pol, pres1 genes and derivatives, and X ORFs, were designed.

Off-target analysis. To verify specificity of the SaCas9/gRNAs generated here, PCR/sequencing analysis of the top predicted off target regions in human genome was performed (Table 1). Sets of primers were designed to amplify these regions followed by subcloning into pCR2.2 TA vector (Invitrogen) and Sanger sequencing.

### Results:

CRISPR/Cas9 design: In silico definition of the twelve most fitting gRNAs. For the eradication of the HBV virus a set of 12 candidate gRNAs was initially selected, targeting the most representative Hepatitis B virus genes. To design these gRNAs the CRISPR designer tool from Benchling, Inc. (benchling.com) was used. The HBV genotype A genome was used as an input sequence and screened for the presence of 20 nucleotide protospacer regions followed by NNGRRT protospacer adjacent motifs (PAMs) which are specifically recognized by SaCas9 endonuclease. The twelve gRNAs shown (FIGS. 1A, 1B) are the gRNAs with the highest “on target” “off target” score. Finally, three gRNAs were chosen based on the most conserved region among ten reported HBV genotypes in NCBI.

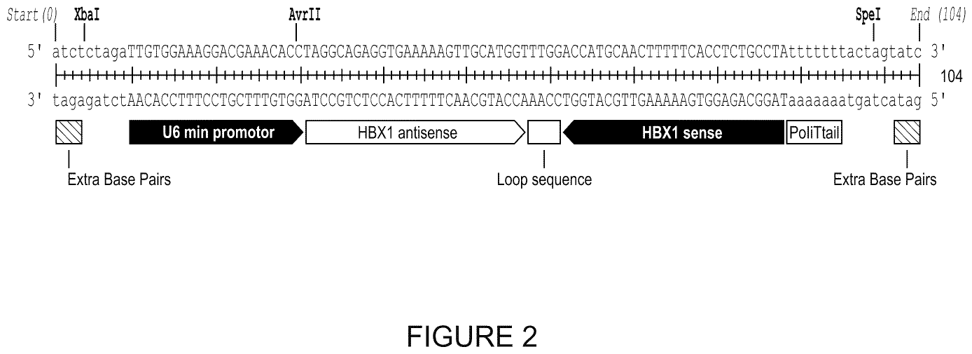

All three gRNAs target the viral polymerase gene (P). Additionally, because of overlap of reading frames, the m1 gRNA targets also the surface protein gene(S), while the m2 and m3 gRNAs target the viral trans-activator protein gene (X). In order to block viral expression in treated cells and to improve gene editing efficiency of SaCas9/gRNAs complexes, a shRNA expressing cassette against X mRNA was added (FIG. 2). All the gRNAs and the shRNA were cloned into a single pX601 vector. The pX601 plasmid is an AAV delivery vector, containing a 1 kb shorter orthologue of the canonical Streptococcus pyogenes Cas9 (SpCas9), derived from Staphylococcus aureus (SaCas9). Shorter SaCas9 gene allows the combining of up to four different gRNA cassettes in a single “all in” vector, without exceeding the restrictive cargo size of AAV, which is around 4.5 kb.

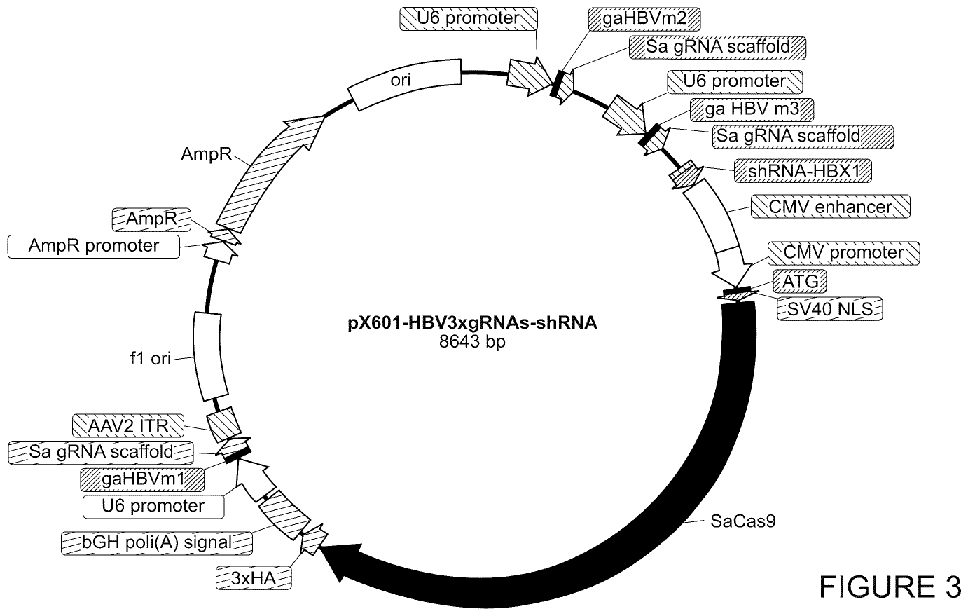

Cloning of the gRNA expressing cassettes targeting HBV genome into pX601-SaCas9-AAV vector and verification final pX601-HBV3xgRNAs-shRNA construct. After the bioinformatics analysis, pairs of sense and antisense oligonucleotides, matching selected target protospacer regions and containing BsaI overhangs on 5′ ends, were ordered, annealed and cloned into a BsaI restriction site, located between U6 promoter and scaffold crRNA sequence in gRNA expressing cassette of pX601 plasmid. To create a multiplex “three in one” gRNAs construct, every single U6-gRNAs cassette was PCR amplified using primers with XbaI/SpeI extensions at their respective 5′ ends. Next the amplicons were cloned, by restriction digestion followed by ligation into pX601-HBVmotif1plasmid XbaI restriction site. The same process was used to add the shRNA-expressing cassette. The final construct is shown in FIG. 3.

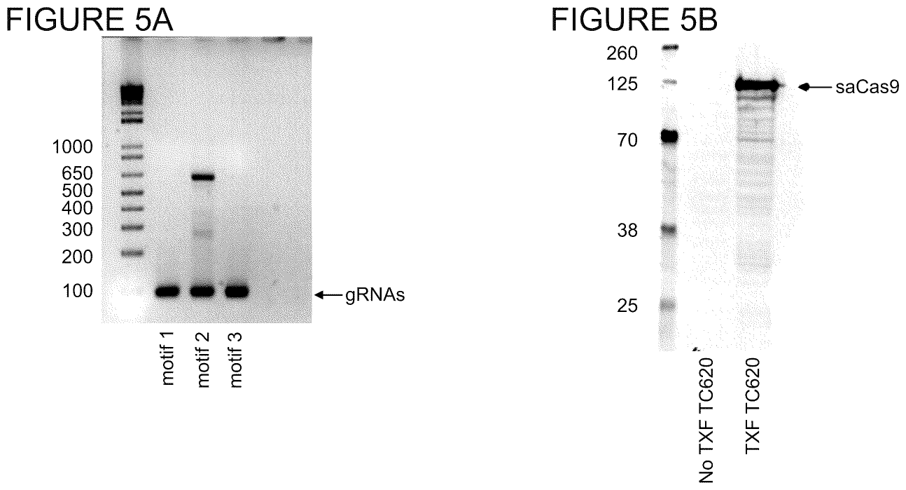

In the next step the final construct was checked to determine whether it was able to express all the components of the SaCas9/gRNA gene editing platform. The pX601-HBV3xgRNA-shRNA construct was transfected into TC620 cells and 48 h later total RNA and proteins were extracted. gRNAs expression was verified in reverse transcription followed by PCR using forward primers specific to each gRNA and scaffold RNA reverse primer (FIG. 5A). To detect NLS-SaCas9-NLS-3xHA protein expression Western blot analysis was performed using HA-tag antibody (FIG. 5B).

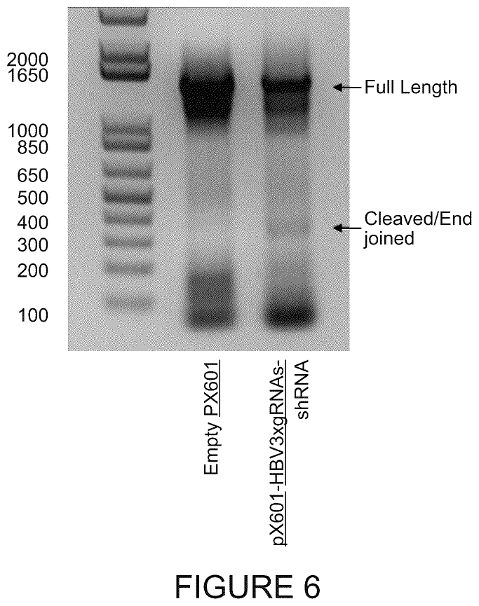

Biological validation of pX601-HBV3xgRNAs-shRNA construct on chronically HBV infected cells (HepG2.2.15). To test the ability of the construct to induce site specific cleavage and excision of HBV genome the chronically HBV-infected HepG2.2.15 cell line was used. 70% confluent cell cultures were transfected with pX601-HBV3xgRNAs-shRNA plasmid, as reported in Materials and Methods. Two days after transfection cells were harvested and genomic DNA was prepared. Next the targeted region of the virus was PCR amplified and resolved by agarose gel electrophoresis. As shown in FIG. 6, two distinct HBV specific PCR products: 1454 bp and 355 bp long were detected. Longer, 1454 bp band corresponds to unmodified full length (in case of control untreated cells) and single cut/end-joined region of HBV genome (in case of SaCas9/gRNAs treated cells). Shorter, 355 bp band represents double cut/end-joined truncated form of viral sequence and is present exclusively in SaCas9/gRNA treated cells.

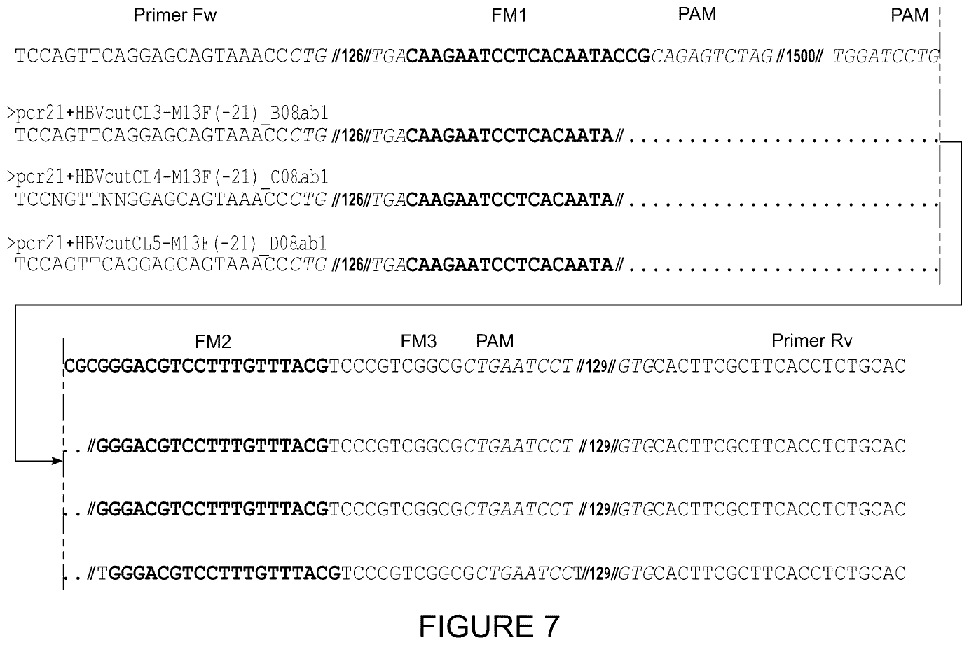

The truncated double cleaved/end-joined band was purified from the gel, cloned and sent for Sanger sequencing. The obtained sequences were aligned using Clustal-Omega software using Hepatitis B genotype D sequence as a reference (FIG. 7). All clones showed perfect CRISPR/Cas9 mediated signature-cleavage three nucleotides from PAM at target sites for motifs 1 and 2. At the target motif 3 no cleavage was detected sinee this gRNA was designed to target exclusively HBV genotype A and in HepG2.2.15 HBV genotype D there are 5 mismatches at this target sites providing additional prove of SaCas9/gRNA specificity (FIG. 7).

Off target analysis. To verify specificity of the excision strategy in targeting the viral genome, analysis of the predicted/possible off targets sites in the human genome was performed. The closest to target sequences hits had at least 3 mismatches (Table 1) making cleavage at these sites highly improbable and inefficient.

With the Primer-Blast tool from the NCBI website, primer pairs were designed for PCR amplification of every genomic region with an off-target score even or above 0.5. After purification and subcloning into a TA vector, amplified predicted off-target regions were sent for Sanger sequencing. No indel mutations were detected in the selected off-target genes.

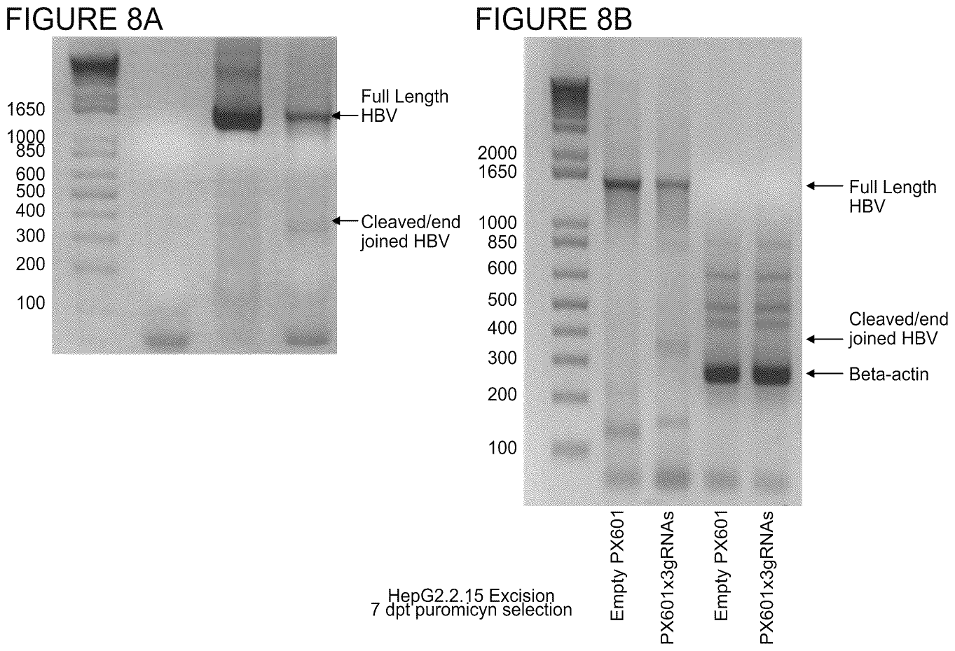

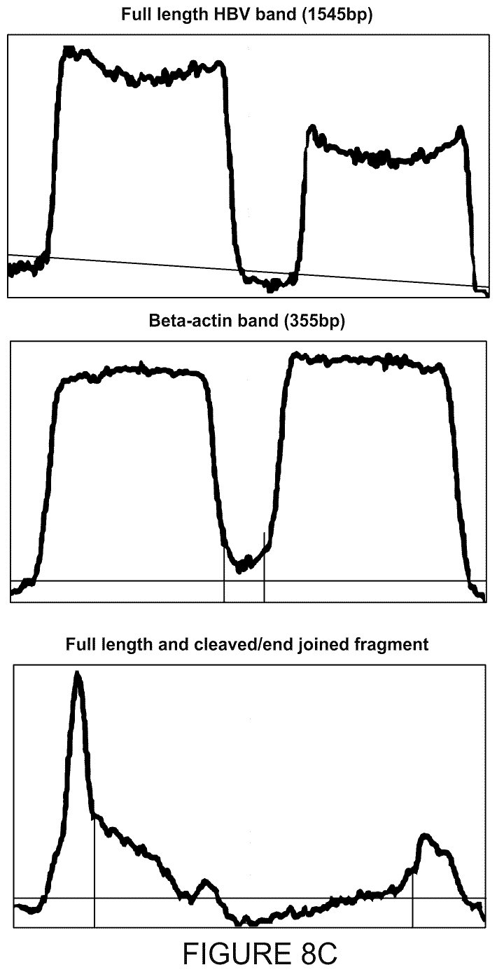

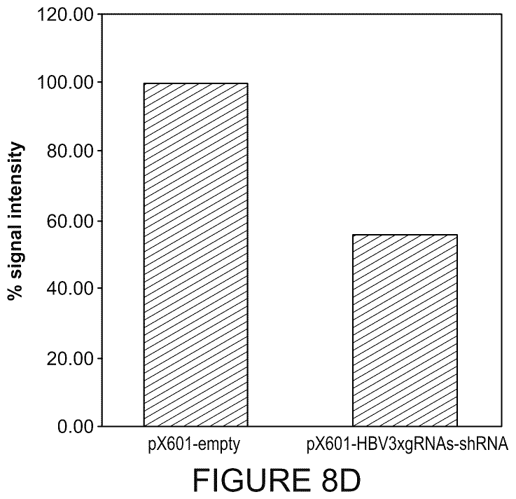

Expression of Cas9/gRNA suppresses Viral replication cycle. To verify the real effectiveness of the construct in blocking viral replication, a further experiment was conducted. HepG2.2.15 cells were transfected with pX601-HBV3xgRNAs-shRNA or control, empty pX601 plasmids. Additionally, pKLV-puro-BFP-empty vector was added to the transfection mixtures in a ratio of 4:1 (2 μg pX601: 0.5 μg pKLV) to permit monitoring of transfection efficiency by BFP fluorescence microscopy and to allow puromycin selection of transfected cells (sinee pX601 AAV vector does not contain any fluorescent label or selection marker). Half of the transfected cells were left untreated and were harvested after 3 days. The rest of the cells were selected for one week under rigorous puromycin regiment (3 μg/ml, medium changed every day) in order to remove untransfected cells and promote stable expression of SaCas9/gRNA in transfected cells. For both populations, the viral integrity and expression was checked at the DNA, mRNA and viral release level. First, genomic DNA was used in standard PCRs with primers specific to targeted region of HBV as was done previously. Again two distinct HBV specific amplification products were detected, full length 1454 bp and truncated 355 bp (FIGS. 8A, 8B). A significant reduction of full length band intensity was noticed in treated cells which is a direct result of SaCas9/gRNA mediated cleavage and degradation of episomal HBV genomes. Additionally, as was shown before, in line corresponding to treated cells characteristic truncated 355 bp long band representing double cut and end-joined viral genome was detected. The ImageJ analysis of band intensities for day 7 timepoint indicated drastic, 50% drop in the level of the full-length HBV DNA in the cells treated with pX601-HBV3xgRNA-shRNA construct (FIGS. 8C, 8D).

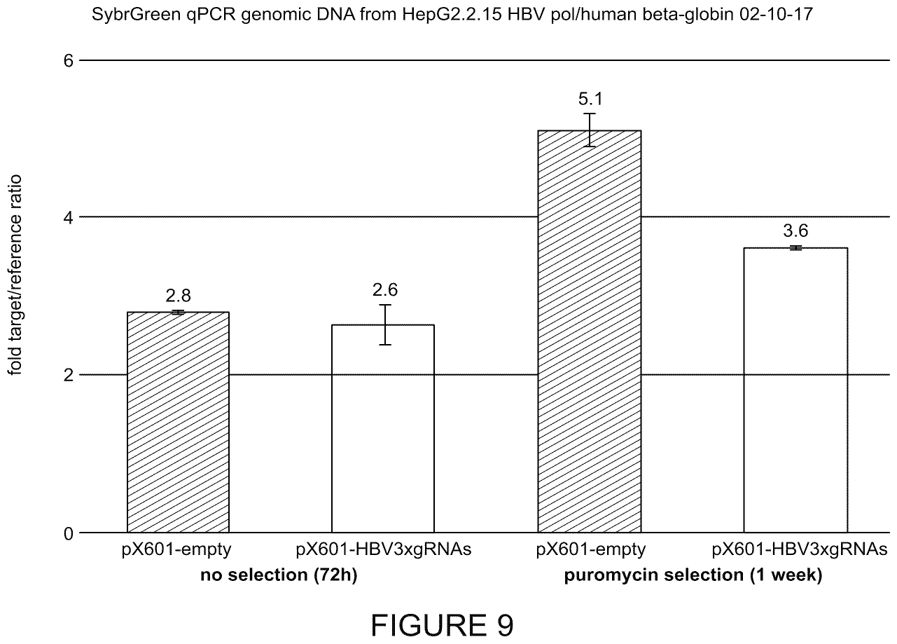

Quantification of intracellular viral DNA levels. To quantify HBV DNA affected by cleavage, a qPCR assay was performed on genomic DNA extracted from HepG2.2.15 treated cells. Using primers specific to HBV pol and reference human beta-globin genes significant drops, close to 30% drop in intracellular viral DNA levels, were detected at seven days post-transfection time point (FIG. 9). The levels of viral DNA at day 3 were lower than at day 7 and only a slight, statistically insignificant, decrease was observed in treated cells for this time point. It is important to note that the primers used in qPCR cannot discriminate between episomal and integrated HBV DNA and they anneal outside of the targeted region of viral genome.

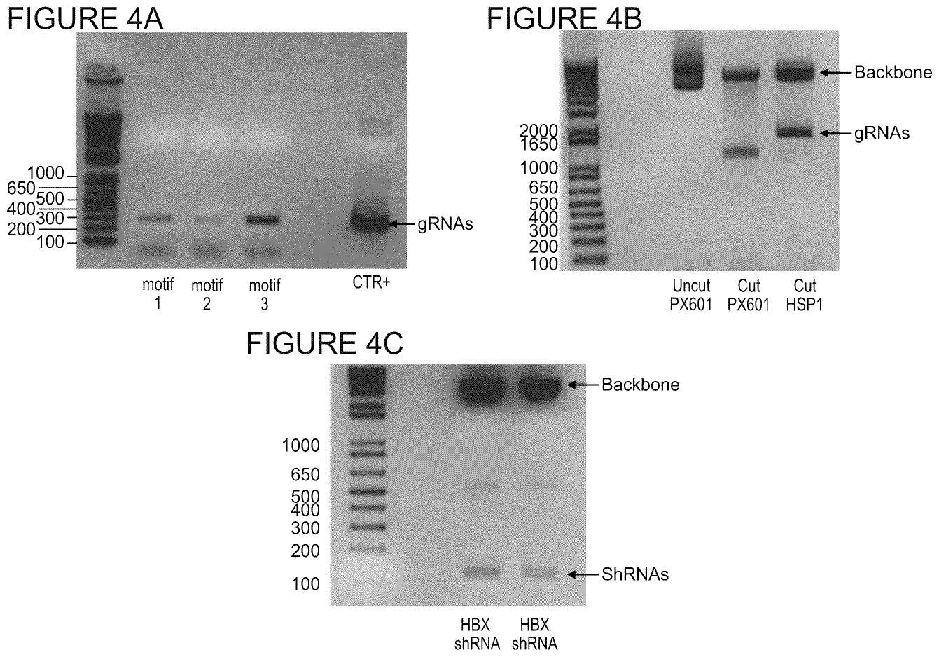

Quantification of viral RNA expression after CRISPR/Cas9 treatment. SaCas9/gRNA mediated cleavage and mutagenesis of HBV genomes in infected cells should result in the decrease of viral RNA levels. To quantify viral RNA levels in treated cells total RNA was extracted and subjected to reverse transcription reaction followed by SYBRGREEN real time PCR assay using primers specific to HBV pol and human beta-actin as a reference. As shown in FIGS. 4A-4C progressive, time dependent reduction of intracellular HBV RNA levels in treated cells was observed. At 3 days after transfection the decrease reached 30% and at 7 days the levels went down to 50% of control, SaCas9/gRNA untreated control.

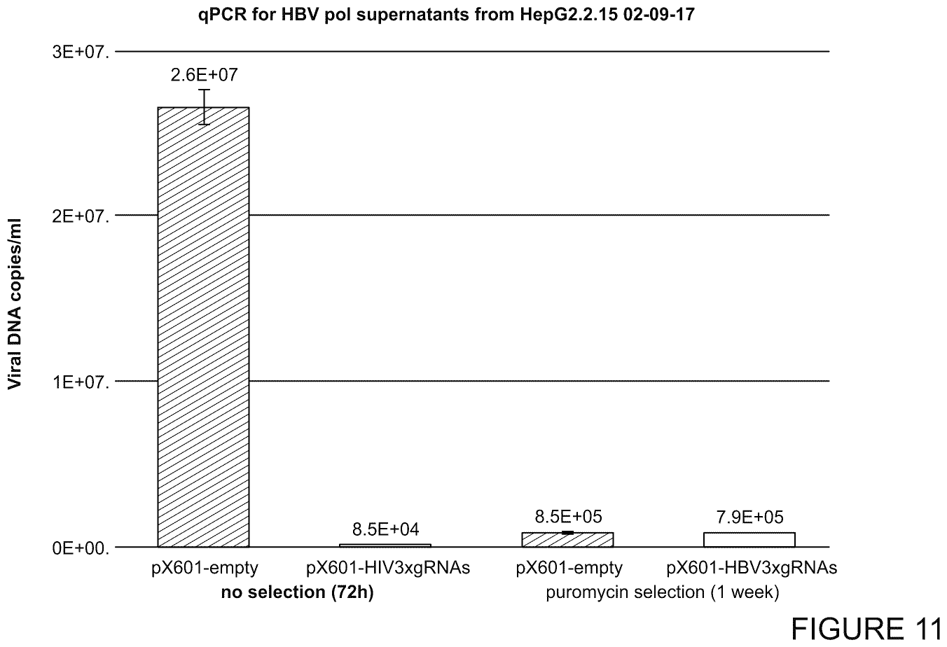

Checking Hepatitis B virus release from treated cells. The final step in viral replication cycle is release of progeny viral particles from infected cells. Viral pregenomic DNA is packaged into viral capsids by interactions with viral core proteins then enveloped and released from infected cells. SYBRGREEN real time PCR was used to measure the levels of viral DNA in supernatants from treated cells which should correspond with viral particles release. As shown in FIG. 11 drastic, more than 95%, depletion of viral DNA levels was observed in the supernatants from treated cells at 3 days post-transfection. At day 7 time point viral DNA levels in supernatants were generally very low and only minimal decrease of was observed in treated versus untreated cells.

### Discussion

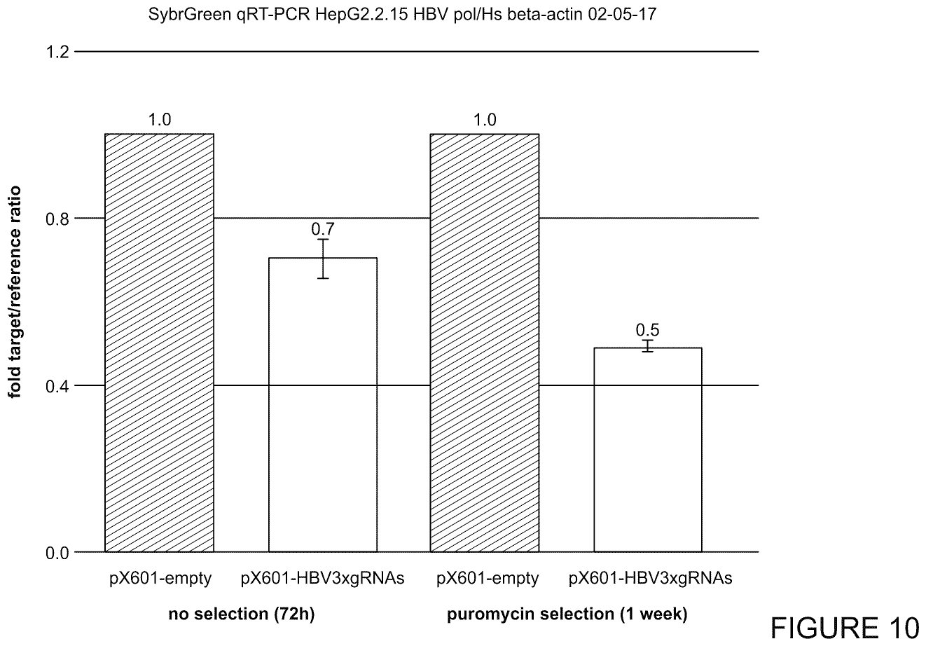

The Hepatitis B virus is still a significant threat for 240 million of people in the world. A novel, CRISPR/SaCas9-based gene therapy is described herein, directed against the persistent HBV DNA genome conserved among all ten HBV genotypes spanning five of the total six viral genes: PreS1, PreS2, S, transactivator X and polymerase. Successful SaCas9/gRNAs-mediated cleavage at these target sites would have different consequences depending on the timing of the cleavage reactions, cellular DNA repair mechanisms and the form of viral genome. Cleavage of episomal cccDNA ordinary leads to its linearization and degradation by cellular exo- and endonucleases. Less frequent end-joining repair and re-circularization results in InDel mutations at the cut sites, in case of single cuts, or excisions/deletions of longer fragments, in case of two or more simultaneous cuts, both resulting in defective viral genomes. In case of much less frequent integrated form of HBV genome, the SaCas9/gRNAs-mediated cleavage would result exclusively in end-joining, InDels and deletions at cut sites. Since PCR primers do not distinguish between episomal cccDNA and integrated HBV genome forms, the products of PCR amplification shown in FIGS. 6 and 8A-8D represent a mixture of both forms. The full length 1454 bp top band consists mostly of episomal cccDNA, sinee it is the predominant form of viral genome present in the infected cells. As mentioned above, the Cas9/gRNAs activity causes fragmentation/linearization and subsequent degradation of cccDNA, which can be observed as a decrease (up to 50% in case of 7 day time point) in the intensity of this band, in the sample of treated cells (FIGS. 8A and 8B lane 2, top bands). On the other hand, the cleavage of the integrated viral genome is promptly repaired by the cellular double strand break repair pathways, mostly by error-prone non-homologous end joining (NHEJ). As a result, full length PCR product corresponding to integrated HBV genome would contain InDel mutations at repaired cut sites, which disrupt or completely block viral gene expression. In case of successful simultaneous cleavage at two sites, the DNA fragment located between them gets edited out leaving truncated defective viral genome, detected as a shorter 354 bp PCR product in SaCas9/gRNAs treated cells (see FIGS. 6 and 8A-8D, line 2). All mentioned above consequences of SaCas9/gRNAs mediated targeting and cleavage of viral genomes in infected cells ultimately culminate in suppression of viral expression. Degradation of viral genomes results in a drop in viral RNA and proteins levels. Additionally, expression from mutated/truncated sequences leads to defective viral mRNAs and proteins as a result of premature transcription terminations and shifted open reading frames. Significant decreases in viral RNA expression levels was observed in SaCas9/gRNAs treated cells as shown in Figure. 10 which mirrors detected depletion of viral DNA. The decrease was greater in cells selected for one week with puromycin which can be explained by the longer period of SaCas9/gRNA expression in the treated cells and death of untransfected (=untreated) cells. The last stage of viral replication cycle is release of the progeny viral particles from infected cells. Here again, consistently with diminished intracellular viral DNA and RNA levels, repression of viral release was detected as measured by qPCR specific to viral DNA in supernatants from gene therapy treated cells. Surprisingly viral DNA level in supernatants of puromycin selected cells was very low in both control (SaCas9 only) and treated cells (SaCas9/gRNAs). Puromycin is aminonucleoside that inhibits translation by disrupting peptide transfer on ribosomes. An inhibitory effect on HBV virion release was not reported before and warrants further studies.

Overall the data herein, provide for the first time proof of successful targeting and cleavage of HBV genome by shorter Staphylococcus aureus derived Cas9/gRNA gene editing platform. Recently other groups reported successful using of canonical SpCas9/gRNA gene editing techniques to target HBV genome (Ramanan et al., 2015). The approach herein, combining triple gRNAs and shorter SaCas9 in single AAV delivery vector provides more robust and is an applicable system to use in clinical settings. To provide a suitable in vivo delivery system the SaCas9/gRNA construct was prepared using as a backbone, plasmid AAV delivery vector pX601. Adeno-associated virus (AAV) vectors are the most commonly used delivery vehicles in vivo, because of their low immunogenic potential, reduced oncogenic risk from host-genome integration, broad-range of serotype specificity, low toxicity and sustained gene expression.

TABLE 1

| Purpose | Name | Sequence |
|---|---|---|
| gRNAs protospacers | gRNA HBV m1 f | CAAGAATCCTCACAATAC |
| | | (SEQ ID NO: 13) |
| | gRNA HBV m1 r | GTATTGTGAGGATTCTTG |
| | | (SEQ ID NO: 14) |
| | gRNA HBV m2 f | GGACGTCCTTTGTTTACG |
| | | (SEQ ID NO: 15) |
| | gRNA HBV m2 r | CGTAAACAAAGGACGTCC |
| | | (SEQ ID NO: 16) |
| | gRNA HBV m3 f | GTCCTTTGTTTACGTCCCGTCGGCG |
| | | (SEQ ID NO: 17) |
| | gRNA HBV m3 r | CGCCGACGGGACGTAAACAAAGGAC |
| | | (SEQ ID NO: 18) |
| Cleavage Detection | HBV 68-89 cut f | TCCAGTTCAGGAGCAGTAAACC |
| | | (SEQ ID NO: 19) |
| | HBV 1476-96 cut r | AGAAGGGGACGAGAGAGTCTC |
| | | (SEQ ID NO: 20) |
| | HBV X1805 f | TCACCAGCACCATGCAAC |
| | | (SEQ ID NO: 21) |
| qPCR analysis | HBV X1896 r | AAGCCACCCAAGGCACAG |
| | | (SEQ ID NO: 22) |
| | HBV pol 2270 f | GAGTGTGGATTCGCACTCC |
| | | (SEQ ID NO: 23) |
| | HBV pol 2392 r | GAGGCGAGGGAGTTCTTCT |
| | | (SEQ ID NO: 24) |
| qPCR references | Hs b-globin f | CCCTTGGACCCAGAGGTTCT |
| | | (SEQ ID NO: 25) |
| | Hs b-globin r | CGAGCACTTTCTTGCCATGA |
| | | (SEQ ID NO: 26) |
| gRNA verification | h/m b-actin f | CTACAATGAGCTGCGTGTGGC |
| | | (SEQ ID NO: 27) |
| | h/m b-actin r | CAGGTCCAGACGCAGGATGGC |
| | | (SEQ ID NO: 28) |
| | gRNA scaffold r | CTCGCCAACAAGTTGACGAGATAA |
| | | (SEQ ID NO: 29) |
| | pX6O1 U6 XbaI f | CTATCTAGAGAGAGGGCCTATTTCCCATG |
| | | (SEQ ID NO: 30) |

1. A composition for eradicating a hepadnavirus in vitro or in vivo, the composition comprising: an isolated nucleic acid sequence encoding a Clustered Regularly interspaced Short Palindromic Repeat (CRISPR)-associated endonuclease and at least one guide RNA (gRNA), the gRNA being complementary to a target nucleic acid sequence in a hepadnavirus genome.

2. The composition of claim 1, wherein the hepadnavirus is hepatitis B virus (HBV).

3. The composition of claim 1, wherein the target nucleic acid sequence comprises one or more nucleic acid sequences in coding and non-coding nucleic acid sequences of the hepadnavirus genome.

4. The composition of claim 1, wherein the target nucleic acid sequence comprises one or more sequences within a sequence encoding structural proteins, nonstructural proteins or combinations thereof.

5. The composition of claim 4, wherein the nucleic sequences encoding structural proteins or non-structural proteins comprise C, X, P, and S nucleic acid sequences or combinations thereof.

6. The composition of claim 1, wherein the gRNA sequence has at least a 75% sequence identity to target nucleic acid sequences comprising C, X, P, and S nucleic acid sequences or combinations thereof.

7. The composition of claim 1, wherein the gRNA sequences have at least a 75% sequence identity to sequences comprising: SEQ ID NO: 1-18, or combinations thereof.

8. The composition of claim 7, wherein the gRNA sequences comprise: SEQ ID NO: 1-18, or combinations thereof.

9. The composition of claim 1, further comprising two or more gRNAs.

10. The composition of claim 9, wherein the two or more gRNAs are complementary to overlapping target sequences, distinct target sequences or combinations thereof.

11. (canceled)

12. (canceled)

13. (canceled)

14. A composition for eradicating a hepadnavirus in vitro or in vivo, the composition comprising: an isolated nucleic acid sequence encoding a Clustered Regularly Interspaced Short Palindromic Repeat (CRISPR)-associated endonuclease and two or more guide RNAs (gRNAs), the gRNAs being complementary to a target nucleic acid sequence in a hepadnavirus genome.

15. The composition of claim 14, wherein the two or more gRNAs are complementary to overlapping target sequences, distinct target sequences or combinations thereof.

16. (canceled)

17. (canceled)

18. An isolated nucleic acid sequence comprising at least a 50% sequence identity to one or more sequences comprising SEQ ID NOS: 1 to 30.

19. The isolated nucleic acid sequence of claim 18, wherein the sequences comprise any one or more of SEQ ID NOS: 1 to 30.


SEQUENCE LISTING (38 sequences)

SEQ ID NO: 1 - DNA, 20 bp, synthetic construct
  misc_feature 1..20: note = Description of Artificial Sequence: Synthetic oligonucleotide
  source 1..20: mol_type = other DNA
caagaatcctcacaataccg

SEQ ID NO: 2 - DNA, 20 bp, synthetic construct
  misc_feature 1..20: note = Description of Artificial Sequence: Synthetic oligonucleotide
  source 1..20: mol_type = other DNA
caaaaatcctcacaataccg

SEQ ID NO: 3 - DNA, 20 bp, synthetic construct
  misc_feature 1..20: note = Description of Artificial Sequence: Synthetic oligonucleotide
  source 1..20: mol_type = other DNA
caagaatcctcacaatacca

SEQ ID NO: 4 - DNA, 20 bp, synthetic construct
  misc_feature 1..20: note = Description of Artificial Sequence: Synthetic oligonucleotide
  source 1..20: mol_type = other DNA
caaaaatcctcacaatacca

SEQ ID NO: 5 - DNA, 20 bp, synthetic construct
  misc_feature 1..20: note = Description of Artificial Sequence: Synthetic oligonucleotide
  source 1..20: mol_type = other DNA
ttgtctacgtcccgtcagcg

SEQ ID NO: 6 - DNA, 20 bp, synthetic construct
  misc_feature 1..20: note = Description of Artificial Sequence: Synthetic oligonucleotide
  source 1..20: mol_type = other DNA
ttgtttacgtcccgtcagcg

SEQ ID NO: 7 - DNA, 20 bp, synthetic construct
  misc_feature 1..20: note = Description of Artificial Sequence: Synthetic oligonucleotide
  source 1..20: mol_type = other DNA
ttgtttacgtcccgtcggcg

SEQ ID NO: 8 - DNA, 20 bp, synthetic construct
  misc_feature 1..20: note = Description of Artificial Sequence: Synthetic oligonucleotide
  source 1..20: mol_type = other DNA
ttgtctacgtcccgtcggcg

SEQ ID NO: 9 - DNA, 20 bp, synthetic construct
  misc_feature 1..20: note = Description of Artificial Sequence: Synthetic oligonucleotide
  source 1..20: mol_type = other DNA
tagacaaaggacgttccgcg

SEQ ID NO: 10 - DNA, 20 bp, synthetic construct
  misc_feature 1..20: note = Description of Artificial Sequence: Synthetic oligonucleotide
  source 1..20: mol_type = other DNA
tagacaaaggacgctcctcg

SEQ ID NO: 11 - DNA, 20 bp, synthetic construct
  misc_feature 1..20: note = Description of Artificial Sequence: Synthetic oligonucleotide
  source 1..20: mol_type = other DNA
tagacaaaggacgctccccg

SEQ ID NO: 12 - DNA, 20 bp, synthetic construct
  misc_feature 1..20: note = Description of Artificial Sequence: Synthetic oligonucleotide
  source 1..20: mol_type = other DNA
taaacaaaggacgctccccg

SEQ ID NO: 13 - DNA, 18 bp, synthetic construct
  misc_feature 1..18: note = Description of Artificial Sequence: Synthetic primer
  source 1..18: mol_type = other DNA
caagaatcctcacaatac

SEQ ID NO: 14 - DNA, 18 bp, synthetic construct
  misc_feature 1..18: note = Description of Artificial Sequence: Synthetic primer
  source 1..18: mol_type = other DNA
gtattgtgaggattcttg

SEQ ID NO: 15 - DNA, 18 bp, synthetic construct
  misc_feature 1..18: note = Description of Artificial Sequence: Synthetic primer
  source 1..18: mol_type = other DNA
ggacgtcctttgtttacg

SEQ ID NO: 16 - DNA, 18 bp, synthetic construct
  misc_feature 1..18: note = Description of Artificial Sequence: Synthetic primer
  source 1..18: mol_type = other DNA
cgtaaacaaaggacgtcc

SEQ ID NO: 17 - DNA, 25 bp, synthetic construct
  misc_feature 1..25: note = Description of Artificial Sequence: Synthetic primer
  source 1..25: mol_type = other DNA
gtcctttgtttacgtcccgtcggcg

SEQ ID NO: 18 - DNA, 25 bp, synthetic construct
  misc_feature 1..25: note = Description of Artificial Sequence: Synthetic primer
  source 1..25: mol_type = other DNA
cgccgacgggacgtaaacaaaggac

SEQ ID NO: 19 - DNA, 22 bp, synthetic construct
  misc_feature 1..22: note = Description of Artificial Sequence: Synthetic primer
  source 1..22: mol_type = other DNA
tccagttcaggagcagtaaacc

SEQ ID NO: 20 - DNA, 21 bp, synthetic construct
  misc_feature 1..21: note = Description of Artificial Sequence: Synthetic primer
  source 1..21: mol_type = other DNA
agaaggggacgagagagtctc

SEQ ID NO: 21 - DNA, 18 bp, synthetic construct
  misc_feature 1..18: note = Description of Artificial Sequence: Synthetic primer
  source 1..18: mol_type = other DNA
tcaccagcaccatgcaac

SEQ ID NO: 22 - DNA, 18 bp, synthetic construct
  misc_feature 1..18: note = Description of Artificial Sequence: Synthetic primer
  source 1..18: mol_type = other DNA
aagccacccaaggcacag

SEQ ID NO: 23 - DNA, 19 bp, synthetic construct
  misc_feature 1..19: note = Description of Artificial Sequence: Synthetic primer
  source 1..19: mol_type = other DNA
gagtgtggattcgcactcc

SEQ ID NO: 24 - DNA, 19 bp, synthetic construct
  misc_feature 1..19: note = Description of Artificial Sequence: Synthetic primer
  source 1..19: mol_type = other DNA
gaggcgagggagttcttct

SEQ ID NO: 25 - DNA, 20 bp, synthetic construct
  misc_feature 1..20: note = Description of Artificial Sequence: Synthetic primer
  source 1..20: mol_type = other DNA
cccttggacccagaggttct

SEQ ID NO: 26 - DNA, 20 bp, synthetic construct
  misc_feature 1..20: note = Description of Artificial Sequence: Synthetic primer
  source 1..20: mol_type = other DNA
cgagcactttcttgccatga

SEQ ID NO: 27 - DNA, 21 bp, synthetic construct
  misc_feature 1..21: note = Description of Artificial Sequence: Synthetic primer
  source 1..21: mol_type = other DNA
ctacaatgagctgcgtgtggc

SEQ ID NO: 28 - DNA, 21 bp, synthetic construct
  misc_feature 1..21: note = Description of Artificial Sequence: Synthetic primer
  source 1..21: mol_type = other DNA
caggtccagacgcaggatggc

SEQ ID NO: 29 - DNA, 24 bp, synthetic construct
  misc_feature 1..24: note = Description of Artificial Sequence: Synthetic primer
  source 1..24: mol_type = other DNA
ctcgccaacaagttgacgagataa

SEQ ID NO: 30 - DNA, 29 bp, synthetic construct
  misc_feature 1..29: note = Description of Artificial Sequence: Synthetic primer
  source 1..29: mol_type = other DNA
ctatctagagagagggcctatttcccatg

SEQ ID NO: 31 - DNA, 104 bp, synthetic construct
  misc_feature 1..104: note = Description of Artificial Sequence: Synthetic polynucleotide
  source 1..104: mol_type = other DNA
atctctagattgtggaaaggacgaaacacctaggcagaggtgaaaaagttgcatggtttggaccatgcaactttttcacctctgcctatttttttactagtatc

SEQ ID NO: 32 - DNA, 1888 bp, synthetic construct
  source 1..1888: mol_type = other DNA
tccagttcaggagcagtaaaccctgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgacaagaatcctcacaataccgcagagtctagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntggatcctgcgcgggacgtcctttgtttacgtcccgtcggcgctgaatcctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtgcacttcgcttcacctctgcac

SEQ ID NO: 33 - DNA, 171 bp, synthetic construct
  source 1..171: mol_type = other DNA
tccagttcaggagcagtaaaccctgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgacaagaatcctcacaata

SEQ ID NO: 34 - DNA, 192 bp, synthetic construct
  source 1..192: mol_type = other DNA
gggacgtcctttgtttacgtcccgtcggcgctgaatcctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtgcacttcgcttcacctctgcac

SEQ ID NO: 35 - DNA, 171 bp, synthetic construct
  source 1..171: mol_type = other DNA
tccngttnnggagcagtaaaccctgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgacaagaatcctcacaata

SEQ ID NO: 36 - DNA, 192 bp, synthetic construct; identical to SEQ ID NO: 34
  source 1..192: mol_type = other DNA

SEQ ID NO: 37 - DNA, 171 bp, synthetic construct; identical to SEQ ID NO: 33
  source 1..171: mol_type = other DNA

SEQ ID NO: 38 - DNA, 193 bp, synthetic construct
  source 1..193: mol_type = other DNA
tgggacgtcctttgtttacgtcccgtcggcgctgaatcctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtgcacttcgcttcacctctgcac